(19) United States
(12) Patent Application Publication
(10) Pub. No.: US 2019/0059550 A1
(43) Pub. Date: Feb. 28, 2019

(54) APPLIANCE FOR TREATING HUMAN KERATIN MATERIALS

(71) Applicant: SEB S.A., Ecully (FR)

(72) Inventors: Franck MANDICA, Francheville (FR); Hélène PERNOT, Lyon (FR); Johan SABATTIER, Mornant (FR)

(21) Appl. No.: 16/061,603

(22) PCT Filed: Dec. 6, 2016
(86) PCT No.: PCT/EP2016/079928

§ 371 Date: Jun. 12, 2018

(30) Foreign Application Priority Data
Dec. 17, 2015 (FR) 1562648

(51) Int. Cl. A45D 34/00; A61N 1/04; A61N 1/30

(52) U.S. Cl. CPC A45D 34/00; A61N 1/0428; A45D 2200/20; A45D 2034/005; A61N 1/303

(57) ABSTRACT

An appliance for treating human keratin materials, includes a treatment head and a handle for holding the appliance in one hand in order to apply the head to the human keratin materials, notably the skin, the appliance having a housing for holding a refill containing a product to be applied, the handle and the treatment head forming an elbow, the housing being oriented substantially in line with the treatment head and open in an opposite direction to the treatment head, the refill being arranged so as to be actuated by the user in order to dispense a metered quantity of product.

This invention concerns appliances for treating human keratin materials, notably the skin, in which a product is applied on the latter from a refill on the appliance.

The invention concerns more specifically, but not exclusively, appliances in which the region treated is subjected to an electric current generated by the appliance, in order to facilitate the action of the product dispensed by the appliance and applied on the keratin materials.

There is a need to permit both the placement and easy removal of the refill and the dispensing of a quantity measured by the user of the product extracted from the refill.

The invention aims to respond to this objective and achieves it by means of an appliance for treating human keratin materials, comprising a treatment head and a handle for holding the appliance in one hand in order to apply the head to the human keratin materials, notably the skin, the appliance having a housing for holding a refill containing a product to be applied, the handle and the treatment head forming an elbow, the housing being oriented substantially in line with the treatment head and open in an opposite direction to the treatment head. The refill is arranged so as to be actuated by the user in order to dispense a dose of product.

Preferably, the refill integrates a pump actuated by pressing the refill when the latter is in place inside its housing.

The invention permits the placement and easy removal of the refill because the housing is accessible from outside of the appliance. In addition, the fact that the refill comprises the pump that is actuated by the user to dispense the product makes the appliance simple to produce and reduces the risk that a pump malfunction may render the appliance unusable, since the refill merely has to be replaced in this case.

Preferably, the refill is mounted on the appliance such that it can move axially in the housing, in order to permit the user to actuate the pump by pressing on the bottom of the refill.

The longitudinal axis of the housing and the longitudinal axis of the handle can make an angle (α) of between 10 and 90°, preferably between 30 and 90°, which makes the appliance ergonomic.

The treatment head can be arranged in order to subject the skin in the treated region to an electric current, the appliance being preferably an electrophoresis appliance.

The housing may lead to the elbow formed between the handle and the treatment head.

The refill may comprise a nozzle for dispensing the product contained inside, and the appliance may be arranged to establish a fluid connection with the dispensing nozzle when the refill is in place inside the appliance, in order to channel the product coming out of the refill to one or more dispensing openings on the treatment head.

The refill preferably protrudes outside of the housing when it is in place inside the latter.

The invention may be better understood by reading the following detailed description of a non-restrictive example implementation, and by examining the attached drawing, in which:

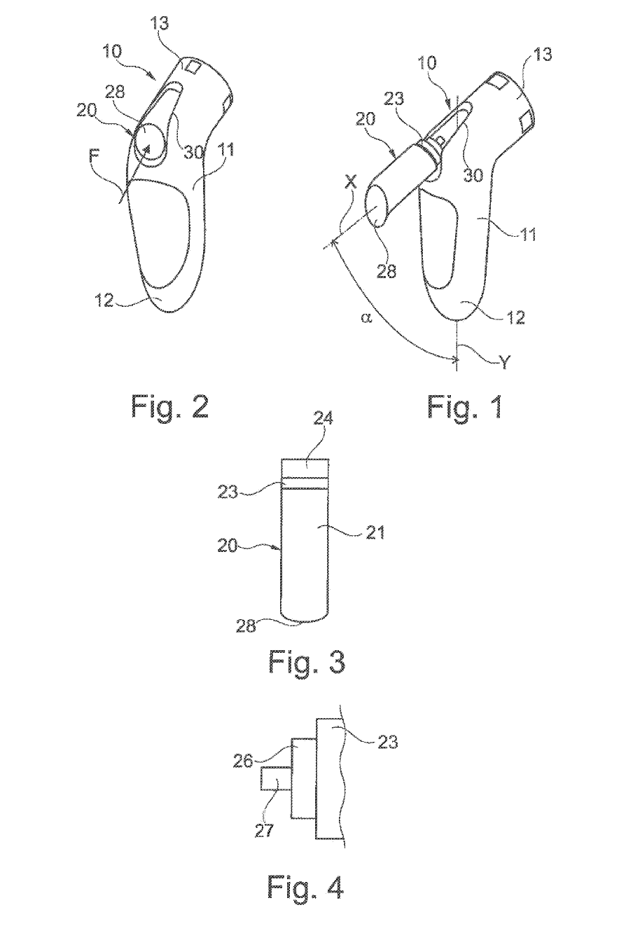

FIG. 1 is a schematic representation of a perspective view of a treatment appliance according to the invention, the refill being on the point of being put into place inside the appliance,

FIG. 2 is a view similar to FIG. 1, the refill having been put into place inside the appliance,

FIG. 3 represents the refill separately, and

FIG. 4 represents a detail of the refill in FIG. 3.

The treatment appliance 10 according to the invention, represented in FIGS. 1 and 2, comprises a case 11 which has an angled shape, with a handle 12 intended to be held in the user's hand, and a treatment head 13 which extends along an axis X which makes an angle α with the longitudinal axis Y of the handle. This angle α is, for example, between 10 and 90°, so as to permit easy handling of the appliance.

The case 11 houses an electronic circuit, not represented, which generates a current within the region treated during operation of the appliance 10, the latter possibly being an electrophoresis appliance. At least one electrode, not visible, may be placed in contact with the product inside the appliance, before the product is dispensed by the treatment head 13. The appliance may also comprise a counter-electrode which is, for example, in contact with the user's hand, so as to permit establishing a circulating current between the electrode and the counter-electrode, within the region treated. Preferably, the product contained in the refill, which is preferably a cosmetic or dermatologic composition, conducts electricity.

The case 11 may also house an electric power source such as one or several non-rechargeable or rechargeable batteries.

FIG. 3 represents separately the refill 20 intended to be used in the appliance and containing the product to be applied.

The refill 20 comprises a body 21 forming a reservoir and a dispensing head 23 which can be equipped before the first use with a protective cap 24, which is then removed before the refill 20 is put into place inside the appliance 10.

FIG. 4 represents a detail of the dispensing head 23, the protective cap 24 having been removed.

The dispensing head 23 comprises a mobile element 26 bearing a dispensing cannula 27 which sealably engages with a corresponding housing provided in the treatment head 13, so that the product dispensed by the refill 20 reaches one or more dispensing openings provided in the treatment head 13.

The element 26 actuates a pump mechanism contained in the refill 20, such a pump possibly being similar to those equipping the cosmetic bottles found on the market.

The refill 20 is held in a housing 30 of the appliance 10, this housing having a longitudinal axis parallel to the longitudinal axis of the head 13, and open at both the top and the back. The housing 30 thus leads to the outer surface of the elbow formed by the junction between the treatment head 13 and the handle 12.

The refill 20 is held in the housing 30 with a possibility of sliding along its longitudinal axis, in order to permit the user, by pressing on the bottom 28 of the refill, to push the latter toward the front and thus cause displacement of the element 26 in relation to the body of the refill, and thus to actuate the pump integrated in the refill. In this way, by pushing according to the arrow F in FIG. 2, the user may dispense a dose of product in the treatment head. When the user stops pushing, the refill is brought backwards by a return spring integrated in the pump. The user can thus dispense as many doses of product as necessary according to the treatment performed, by acting on the refill 20.

The appliance is advantageously arranged so as to detect a need for product and to alert the user by issuing a visual, audible or vibrating signal by means of one or more indicators, for example, not visible in the figures. Thus, whenever a signal asks the user to dispense a dose of product, the user may act on the refill 20 as explained previously.

The need for product is determined, for example, by measuring impedance of the circuit between the electrode and the counter-electrode, which can provide information about the quality of the contact of the appliance with the keratin materials treated, insufficient electrical conduction possibly being representative of an insufficient quantity of product at the interface between the treatment head and the keratin materials treated.

The bottom 28 of the refill 20 can be given a shape with a non-circular contour, notably elliptical, the bottom 28 being able to extend in a plane which is not perpendicular to the longitudinal axis X of the treatment head 13, so as to facilitate pushing of the bottom 28 by the user, notably with the thumb. The bottom 28 may in particular extend substantially according to a plane parallel to the longitudinal axis Y of the handle, when the refill 20 is in place.

The appliance 10 and the refill 20 are advantageously configured such that in this case the refill 20 is arranged in a manner oriented in the housing 30, with for example the large axis of the ellipsis defining the bottom 28 of the refill 20 oriented in a plane coplanar with the longitudinal axis Y of the handle.

The invention is not limited to the example which has just been described.

In particular, the refill may comprise a flexible wall on which the user presses to dispense the product.

1. An appliance for treating human keratin materials, comprising: a treatment head; a handle for holding the appliance in one hand in order to apply the head to the human keratin materials, notably the skin, and a housing for holding a refill containing a product to be applied, the handle and the treatment head forming an elbow, the housing being oriented substantially in line with the treatment head and open in an opposite direction to the treatment head, the refill being arranged so as to be actuated by the user in order to dispense a dose of the product.

2. The appliance according to claim 1, wherein the refill integrates a pump actuated by pressing the refill when the refill is in place inside its housing.

3. The appliance according to claim 2, wherein the refill is mounted on the appliance such that the refill is movable axially in the housing, in order to permit the user to actuate the pump by pressing on the bottom of the refill.

4. The appliance according to claim 1, wherein a longitudinal axis of the housing and a longitudinal axis of the handle make an angle (α) of between 30 and 90°.

5. The appliance according to claim 1, wherein the treatment head is arranged in order to subject the skin in the treated region to an electric current.

6. The appliance according to claim 1, wherein the housing leads to the elbow formed between the handle and the treatment head.

7. The appliance according to claim 1, wherein the refill comprises a dispensing nozzle for dispensing the product contained inside and the appliance is arranged to establish a fluid connection with the dispensing nozzle when the refill is in place inside the appliance, in order to channel the product coming out of the refill to one or more dispensing openings on the treatment head.

8. The appliance according to claim 1, wherein the refill protrudes outside of the housing when the refill is in place inside the housing.
